(19) United States
(12) Patent Application Publication
(10) Pub. No.: US 2015/0209615 A1
(43) Pub. Date: Jul. 30, 2015

(54) Zoning Method of Processing Threshold and Metabolic and Heart Rate Training Data and Sensors and Apparatus for Displaying the Same

(71) Applicant: Sally Edwards, Sacramento, CA (US)

(72) Inventor: Sally Edwards, Sacramento, CA (US)

(21) Appl. No.: 14/165,118

(22) Filed: Jan. 27, 2014

(51) Int. Cl. A63B 24/00; A61B 5/22; A61B 5/00; A61B 5/024

(52) U.S. Cl. CPC A63B 24/0075; A61B 5/02438; A61B 5/222; A61B 5/743; A61B 5/6831

(57) ABSTRACT

A system for increasing the fitness level of a fitness enthusiast. The system includes a personalized set of intensity zones corresponding to increasing levels of exercise intensity. The personalization of the system is accomplished through the determination of a first and second anchor point based on changes in the rate of change of an individual's heart rate as exercise stress is increased. Values for a plurality of zones are determined based on at least one of said anchor points. Each zone corresponds to a multiplier that when factored in to the amount time spent in each zone by the individual, yields a total training load value for the individual as the individual exercises.

### BACKGROUND OF THE DISCLOSURE

1. Technical Field of the Disclosure

The present invention relates in general to processing and displaying training data of an individual in real time. More specifically, the present disclosure relates to a method of collecting, analyzing, storing, reporting and processing training activity data.

2. Background and Description of the Related Art

Training and exercise is an extremely important component of maintaining a healthy lifestyle. Regular physical exercise can help one maintain a healthy weight and can reduce the risk of cardiovascular disease, type 2 diabetes, metabolic syndrome, depression, arthritis, and certain types of cancers. Exercise also increases one's overall energy and one's mental health and mood. Exercise and physical activity burns calories, increases blood flow, improves muscle and bone strength, and helps the heart and lungs work more efficiently.

Objectively measuring one's physical excursive can be difficult. The level of physical exertion it takes to complete an exercise and even the caloric cost of that exercise can vary depending on one's level of fitness and many other factors. For example, in something called the training effect it is well known that as one's aerobic system becomes more efficient (fitter) over time, performing the same activity (such as running one mile) will require less exertion. For these and other reasons it can be difficult to gauge the true intensity of one's workout or when to increase the intensity and how much to increase it by. Monitoring one's heart rate can give an idea of how vigorously an individual is pushing himself or herself, thereby helping the individual exercise at the right pace (and more efficiently).

All mammals, including humans, rely on a heart in order to circulate blood, which carries oxygen and other necessary molecules and nutrients throughout the body. A common measurement number of the effort exerted by a human at rest or during activity is heart rate. Heart rate is the speed at which a heart beats or contracts, generally measured as heart beats per unit time, most commonly in units of beats per minute (BPM). Is has long been known that heart rate is an indicator of exercise intensity level or exercise effort leading to physical stress. This is due to the fact that active muscles require more oxygen than inactive muscles, and consequently the heart must beat more frequently to circulate oxygen and nutrient-carrying blood during periods of exercise.

Consequentially, measuring heart rate is one of the primary and most popular means of monitoring the effect and intensity of exercise. While there are many different methods that may be used to measure physiological status during exercise, such as thermometers, metabolic meters, respiratory monitors, stress monitors, and power meters, measuring heart rate allows one to monitor a blend of physiological status markers non-invasively and inexpensively for the user, and is thus the most frequently used means of measuring physiological status. Heart rate may be used to track improvement and performance over time, also called adaptations and trends, and to quantify and improve both the quality and benefit of exercise leading to improvements in health, fitness, and performance.

One popular conventional method for monitoring heart rate, outside of a clinical setting, involves recording heart rate data based on a percentage or fraction of one's maximum heart rate. Maximum heart rate (“HRₘₐₓ”) is the maximum number of heartbeats per unit time, typically one minute, which an individual can achieve through exercise without incurring severe health problems. HRₘₐₓ is generally measured in units of beats per minute (“BPM”), and is generally reached only during the most strenuous of physical activities. It is advised that one always approach HRₘₐₓ with caution. HRₘₐₓ is generally loosely inversely related to a person's age, but it may vary greatly from person to person depending up many physiological factors. The most accurate measurement of HRₘₐₓ is attained by direct measurement of heart rate using a heart rate monitor during a cardiac stress test.

In recent years, low cost and accurate heart rate monitors that record real time data regarding the user's heart rate have become very affordable. Consequentially, even casual exercisers and trainers now commonly use heart rate monitors. There are many types of heart rate monitors currently on the market. One popular type comprises a two-part system including a chest sensor/transmitter to detect the electrical activity of the heart, and a receiver that works in conjunction with the chest sensor/transmitter and detects signals sent by the chest sensor/transmitter. The sensors on the chest strap generally comprise electrodes and are placed against the user's skin, thereby sensing the electrical fluctuations (changes or responses) of the heart present during each heartbeat. The chest strap or transmitter belt transmits a wireless signal to a receiver. While some cardio-machines and other workout machines may receive the transmission, receivers can also be built into mobile phones and mobile devices or build into watches or other displays accessible to the user during exercise. Heart rate monitors allow detailed and reliable measurements to be continuously taken at times where it would otherwise be difficult for one to record heart rate any other way, such as by holding a finger to the carotid artery and counting for sixty seconds or other palpation methods. Additionally, heart rate monitors and devices and consoles on cardio machines commonly store heart rate data over the course of an entire workout or exercise session.

Advanced heart rate monitors now include features such as average heart rate over a period of time, calories burned (generally calculated based on heart rate over time, age, and the user's weight), time spent in a specific heart rate zone, and the capability to output detailed graphs via an internal display or to other computing devices such as mobile phones, tablets and desktop or laptop computers. Through the use of heart rate monitors, the user is provided with detailed information regarding his or her cardiovascular system's response over time. The explosion in the use of these heart rate monitoring devices has led to the development of many different schemes for determining “zones” of heart rate ranges that produce varying effects on the human body, wherein the “zones” have historically been ranges of BPM based on the user's HRₘₐₓ.

Another type of heart rate monitor, apart from the two-part system described above, is commonly known as a “contact monitor.” This type of heart rate monitoring device uses sensors that are embedded in cardiovascular equipment such as the handles on treadmills, elliptical machines, stationary bicycles, stair climbing machines, rowing machines, and other similar equipment. These embedded contact heart rate monitors require the user's hand to contact a metallic plate in order to allow a sensor embedded within to measure heart rate, which is then generally displayed on a screen for the user. Contact heart rate monitors provide the same heart rate data as other monitors, generally in BPM, and often offer many of the advanced functions described above.

Yet another type of heart rate monitor is what is known as an optical heart rate monitor. These monitors do not utilize a chest strap and may instead detect heart rate information from a device that is secured to the user's wrist or forearm. The system utilizes an optical sensor and light source to gather information about the wearer's pulse. The same device that monitors and detects the pulse can function as a display for the user.

The nuances that distinguish one heart rate monitor from another are found in the methodologies employed to calculate or assess, often in real time, the fitness level, at-the-moment heart rate or overall exertion of a user. As indicated above, some of the simplest means of doing so involve determining which “zone” a user's heart rate is in at any given moment. For instance, one conventional though now outdated rule of thumb describes a zone that includes a range of heart rates between 70% and 85% of one's HRₘₐₓ. It was believed that within this zone one would achieve maximal fitness gains.

Conventionally and most commonly, a user's HRₘₐₓ was determined based solely upon the user's age by comparing a person's age with the HRₘₐₓ predetermined for that age—generally found on a chart or a simple calculation. Due in large part to marketing measures by many companies, it is still generally assumed by many individuals that as one gets older, one's HRₘₐₓ drops by a set amount per year of aging, and that therefore the intensity of one's target heart rate zone should drop accordingly. This approach to determining HRₘₐₓ suffers from several drawbacks including (1) each person's HRₘₐₓ is specific to that person and it may be inaccurate to assume a HRₘₐₓ can be based solely on the person's age, (2) there are great benefits to be reaped by maintaining a person's heart rate in the heart rate zones above and below the zone of 70% to 85% HRₘₐₓ, and (3) the positive effects of maintaining a person's heart rate in a certain zone is further enhanced and affected by the amount of time spent in that zone.

In 1993 the Applicant pioneered a now-popular system used for finding a training load based on each individual's HRₘₐₓ (hereinafter the “Heart Zones Training” system) as calculated based on sub-max or max testing protocols. The “Heart Zones Training” system still emphasized the importance of zones calculated based on HRₘₐₓ, but did not presume to assign a max heart rate based solely on an individual's age.

Although the Heart Zones Training system offered several benefits including recognizing the greater fitness (stress or caloric expenditure or training load) benefits imparted by a user spending exercise time in the higher zones, and zones that were individualized to each user, the Heart Zones Training system still relied on static not dynamic zones, meaning they would not adjust due to a user's increasing or decreasing fitness level and cardiovascular health over time unless new sub-max or max heart rate testing was performed again. An additional downside to the Heart Zones Training system was that the zones were calculated in a linear manner and thus did not take into account the fact that increments in exercise intensity above several different points or effort levels tend to have an increasing and then an exponential stress effect on the human body.

Another methodology of acquiring training load data is referred to as the “Training Effect.” This methodology categorizes training intensities on a scale of 1 through 5 and based on a person's HRₘₐₓ measured for a specific sport or exercise. Based on this HRₘₐₓ, which must be first calculated by the fitness enthusiast using any number of a variety of methods, zones of <60% max heart rate, 60-70% max heart rate, 70-80% max heart rate, 80-90% max heart rate and 90-100% max heart rate have been developed. The lowest level is described as having mostly restorative benefits and being capable of producing benefits in basic fitness, especially after a long break from exercise. The Training Effect methodology further states that the majority of cardiovascular training should occur within the 60-70% zone, that exercising in the 70-80% zone will improve one's agility and efficiency in movement, and warns against overtraining in the 80-90% zone. As the intensity in each zone increases, the Training Effect method also describes the body's inability to process built up lactic acid.

It is now known that HRₘₐₓ varies tremendously from person to person, even in persons of the same age and the same fitness level. HRₘₐₓ also varies from sport to sport and exercise activity to exercise activity and even as a user performs the same activity but at a higher or lower elevation. A person's HRₘₐₓ in one sport or exercise activity can be as much as 20 BPM lower than that same person's HRₘₐₓ in another sport or exercise activity. Hence, a person's heart rate as compared to that person's own baseline values for the sport or exercise activity engaged in provides a much better gauge of cardiorespiratory and cardiovascular activity than a comparison against a static set of values to be used for all users regardless of activity. The drawback to this method is that in order to avoid comparison against a static set of values for all users across all activities, a person must determine his or her own HRₘₐₓ for the particular sport or exercise in which he or she is engaged, as described above with regard to the Heart Zones Training system. Although comparison against a static set of values for all users might be easier for some fitness enthusiasts, the benefits of comparison to one's own base line values are worth the extra effort for most people.

There is thus a need for a comprehensive and personalized system for monitoring the effects of training and activity in dynamic zones taking into account the fact that increments in exercise intensity above two certain points, metabolic thresholds, tend to have curvilinear and then exponential effects on stress in the human body.

It is thus a first objective of the present invention to provide a training load curve that moves from linear to curvilinear to exponential as intensity increases.

A second object of the present invention is to provide a system that will process and display training data of an individual in real time.

A third objective of the present invention is to provide a system that would allow an individual exercising to see real-time feedback regarding their level of physical exertion.

A fourth objective of the present invention is to provide a system that would allow an individual exercising to see the measurement and the details regarding the total physical exertion amount over time.

A fifth objective of the present invention is to collect, analyze, store, report and process training activity data.

These and other advantages and features of the present invention are described with specificity so as to make the present invention understandable to one of ordinary skill in the art.

### SUMMARY OF THE DISCLOSURE

To minimize the limitations found in the prior art and to minimize other limitations that will be apparent upon the reading of the specification, the preferred embodiment of the present invention provides a system and method for processing and displaying training data of an individual in real time. More specifically, the present disclosure relates to a method of collecting, analyzing, storing, reporting and processing training load data and exercise stress measurements.

Utilizing a heart rate monitor with specific algorithms that collect data regarding a training load curve that moves from a first linear rate, a second linear rate to an exponential rate of increase as intensity increases, the system provides in a preferred embodiment visible feedback to the user. In other embodiments feedback is not provided directly to the user but instead is sent for collection by other devices.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention. Thus, the drawings are generalized in form in the interest of clarity and conciseness.

FIG. 1 shows a top view of a first embodiment of the invention;

FIG. 2 shows a top view of a second embodiment of the invention;

FIG. 3 shows a perspective view of a heart rate monitor chest strap;

FIG. 4A shows a chart depicting an exemplary training regimen at week 1.

FIG. 4B shows a chart depicting an exemplary training regimen at week 2.

FIG. 5A shows a chart depicting an exemplary training regimen at week 7.

FIG. 5B shows a chart depicting an exemplary training regimen at week 7.

FIG. 6 depicts a chart showing three zones and various details about each zone;

FIG. 7 depicts a chart in accordance with the present invention, showing a simplified model of the zones in FIG. 1, compiled into zone groupings;

FIG. 8 depicts a chart in accordance with the present invention, showing the floor and ceiling heart rates for an individual having an anchor point of 150 BPM;

FIG. 9 depicts a chart showing a multiplier associated with a plurality of zones, wherein for the zones preceding a threshold zone a substantially linear increase in multiplier is depicted and wherein for the zones following a threshold zone a substantially exponential increase in multiplier is depicted; and

FIG. 10 depicts a chart showing heart rate on the X axis and exercise intensity, such as ventilation quantity, on the Y axis wherein an exponential cost is shown after T2.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

In a preferred embodiment, the system captures data regarding a user's heart rate from a heart rate monitor worn by the user. For this process it is to be understood that although heart rate is a function of time and can be described according to any time frame, in a preferred embodiment of the invention the individual's rate in BPM is used. A heart rate monitor is a personal monitoring device that allows an individual to measure his or her heart rate in real time or record the heart rate for later study. The specific embodiment of the device may vary from chest electrodes to straps worn around the chest or hand to optical devices that can detect a heart rate and that are either in contact with the user or not in contact with the user. The simplest current devices utilize a dedicated heart rate monitor device (such as an electrical signal detector worn around the chest) and second component in the form of a receiver and display, although in some embodiments such as a wrist strap monitoring device the two can be integrated as one. In the preferred embodiment of the invention, the user wears a chest strap that monitors heart rate and transmits heart rate data to a wristwatch that is either worn or placed on a bicycle for easy viewing while the bicycle is in use. In an alternative embodiment a foam adapter for placement on the handlebars of the user's bicycle is provided. The foam adapter (not shown) provides an attachment point for the heart rate monitor display. In yet another alternative embodiment of the invention the device is worn on the wrist and both detects pulse optically or electrically through the wrist and displays and records data regarding said pulse (that is, heart rate). In still yet another embodiment the system detects pulse optically via an external video camera without the need skin contact.

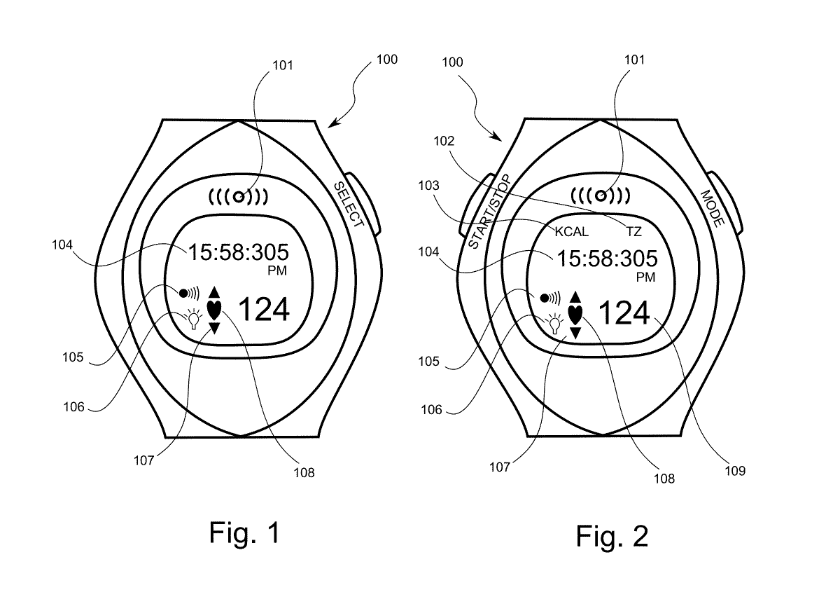

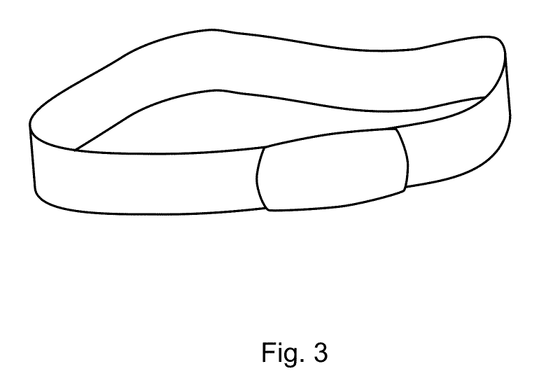

The specific embodiment of the heart rate tracker according to the present invention may take any number of forms, and two exemplary wrist-mounted embodiments are shown as FIGS. 1-3. Turning first to FIGS. 1 and 2, a top view of a two exemplary embodiments of the wrist-mounted portion 100 of the present invention are shown. This wrist-mounted portion may comprise metal, plastic, composite, crystal, or other materials as known in the art, and any combination thereof. The wrist-mounted portion of this embodiment generally comprises a microprocessor as known in the art, a receiver and/or transceiver, a display, and at least one control. The wrist-mounted portion further comprises a LED-flashing indicator 101. The LED-flashing indicator may be configured to illuminate in two or more colors and in a preferred embodiment three colors, or in only one color. The wrist-mounted unit may further comprise a speaker and may be waterproof or water resistant.

The display may be configured to display at least one of a target zone 102, calories burned 103, the time 104, an out-of-zone alarm 105 to indicate to the user that he or she is outside the target zone, an LED mode indicator 106, an in-the-zone indicator 107 to indicate to the user that he or she is in the zone, an indicator of when the device is measuring heart rate 108, and the user's current heart rate 109.

Typical functions of a heart rate monitor display as disclosed in this application may include current heart rate display and data capture, average heart rate display and data capture, peak heart rate display and data capture, time spent within a training zone and data capture, time spent above a training zone and data capture, time spent below a training zone and data capture, time spent below T1 (defined in more detail below), time spent between T1 and T2 (fined in more detail below), time spent above T2 (fined in more detail below), calories burned and data capture, stop watch and time of day and date, month, year, information. In a preferred embodiment the heart rate monitor takes the form of a wristwatch that is water resistant, includes a changeable battery and multiple means for interfacing with the user. For instance, the heart rate monitor may flash one of three colors to the user as disclosed below, or may flash one of three different tonal beeps to the user, each different tone indicating the entering of a different zone.

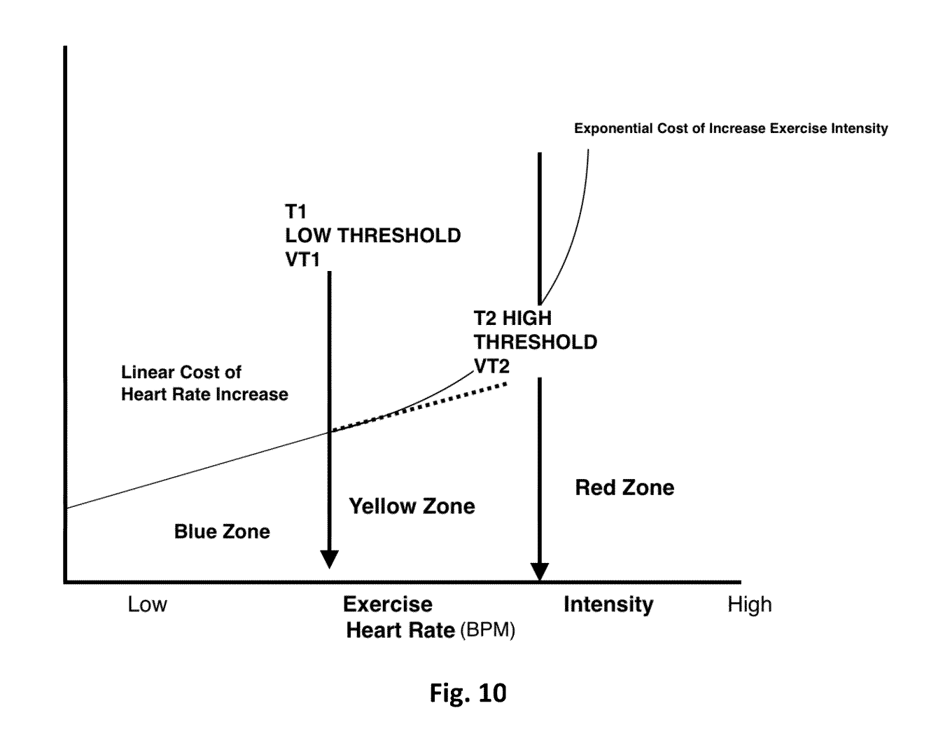

Conventional systems have traditionally used an individual's HRₘₐₓ as one anchor point upon which all zones were dependent. The target zone of the Applicant's system can be defined as the zone between a low threshold level referred to as “T1” (See FIG. 10) and a high threshold level referred to as “T2”, also known as a first anchor point and a second anchor point, respectively. These anchor points are determined for each individual and for each individual sport, and both the first and second anchor points are below HRₘₐₓ. It is noted that VO₂ max is the maximum amount of oxygen an individual's body is capable of using in one minute. Traditionally, the value is recorded in milliliters of oxygen per kilogram of weight per minute. Athletes tend to have higher VO₂ max values, and in general, one's VO₂ max and heart rate at T1 and T2 will increase with increased fitness.

As noted above, the Applicant's system utilizes two anchor points. T1 and T2, where each anchor point signifies a change in the rate of exercise stress and metabolic response to that intensity as intensity increases. For purposes of this application, the system can be said to utilize a first and second anchor point or the synonym first and second threshold level or threshold point.

One means for determining the high threshold level T2 marks T2 as that heart rate corresponding to the intensity of exercise that cannot be sustained due to lactic acid accumulation in the blood and other physiological mechanisms. There are a number of metabolic markers that when measured indicate this changeover point from aerobic to nonaerobic, including but not limited to lactic acid level, oxygen exchange, and carbon dioxide exchange. For instance, lab testing of blood lactate levels may be performed with the understanding that a typical blood lactate level is 2 millimoles per liter, and anaerobic threshold is commonly held to have occurred when lactic acid concentration reaches 4 millimoles or greater per liter.

One's body can cross the second anchor point (T2) because the individual is incapable of using any additional oxygen per unit time. Beyond T2, some carbohydrates are processed without the use of oxygen, ultimately leading to the build up of lactic acid in the individual's body. At this point and above, the energy needed by the body to maintain the given intensity level exceeds the oxidative process capabilities of the body, and nonaerobic or anaerobic metabolism begins. Since lactate is a salt substance produced from lactic acid, which itself is a product of muscle contraction, measurement of an individual's lactate level indicates the point at which one's metabolism crosses over from aerobic to nonaerobic. The heart rate of the individual at this dividing point between aerobic and anaerobic metabolism is the threshold rate.

Another means for lab testing an individual's threshold heart rate number is through testing the ventilatory threshold. In field-testing it is estimated by a shift in breathing patterns while in lab testing it can be determined by the ratio of carbon dioxide expired and oxygen inspired.

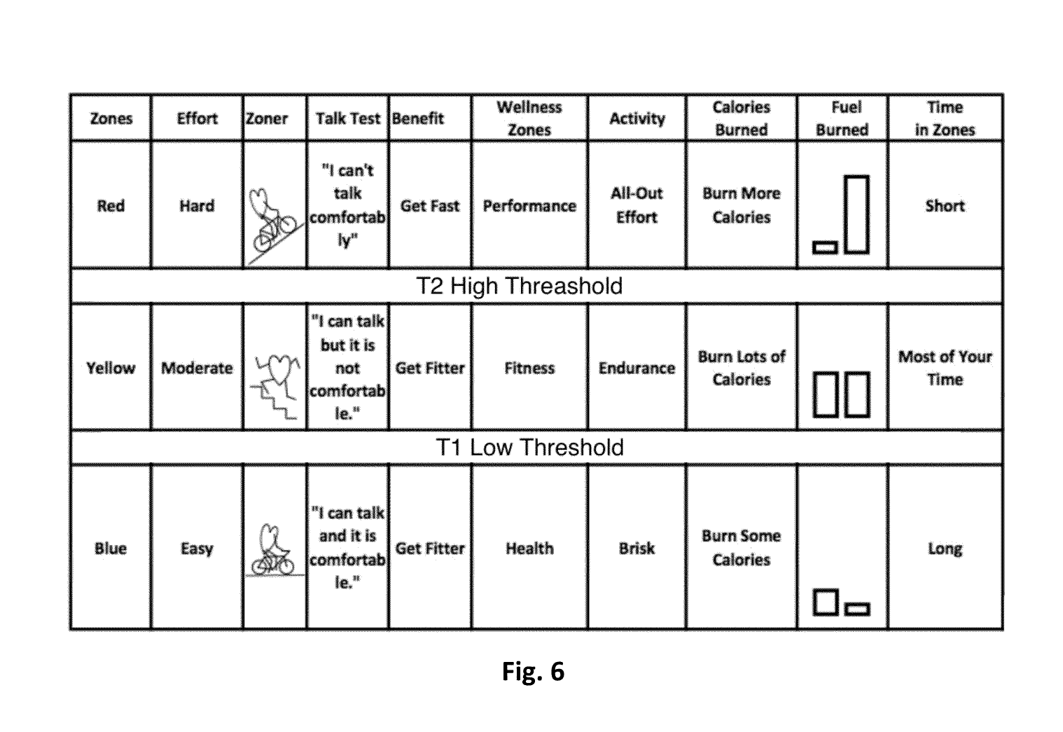

Another system for estimating the threshold rate is the commonly known as the Talk Test, which is based on the premise that at a certain point an large enough increase in ventilation requirements prevents the subject from speaking comfortably. Carl Foster, Ph.D. a professor at the University of Wisconsin, first devised one of several different protocols for a Talk Test. The Talk Test defined the point at which the exercise intensity of an individual is sufficient as the point where the individual can no longer comfortably recite aloud a standard paragraph (commonly the Pledge of Allegiance) at a reasonable rate without pausing for breath. This point is the ventilatory threshold point. At this crossover intensity level, the ventilatory demands are greater than the ability of the oxygen delivery system to keep up. At this point ventilation rate increases dramatically. See FIG. 6.

Although various means may be used to determine one's T1 and T2 anchor points, in all cases the anchor points are sport and exercise specific and are dynamic, that is, they change with an individual's fitness level, nutritional intake, and environmental factors, such as temperature, humidity or altitude. They do not necessarily change with an individual's age.

Using any of the above methods to determine anchor points, the data is then imported to a chart such as that shown in FIG. 6. In FIG. 6, attention is brought to T1 and T2 thresholds, or anchor points. In this very simplified chart the entire spectrum of exercise intensity of an individual is condensed down to just three zones--blue, yellow, and red.

The Applicant's system further uses the principle of weighted zones, that is, each of the zones has associated with it a multiplier. Higher zones demand more oxygen and metabolic nutrients from the body and hence higher multiplier is associated with them. The multiplier used in the Applicant's system reflects the fact that increments in exercise intensity above a certain point tend to have curvilinear or more exponential effects on stress in the human body. The user's heart rate response above a certain point, defined by the Applicant as the “second threshold or high threshold point,” increases exponentially, as shown best at FIG. 10.

Returning to FIG. 6, the row with the word blue at left corresponds to zone 1, the row with yellow at left corresponds to zone 2, and the row with red at left corresponds to zone 3. As described below, the system may calculate total work effort by multiplying the amount of time spent in each zone by the zone number. In this exemplary embodiment, if an individual spent 5 minutes within each zone, a number of 25 would be derived (5 minutes×zone 1+5 minutes×zone 2+5 minutes×zone 3.) In practice, this simplified version is not preferred, however, it provides a basic understanding of the concept described in more detail below.

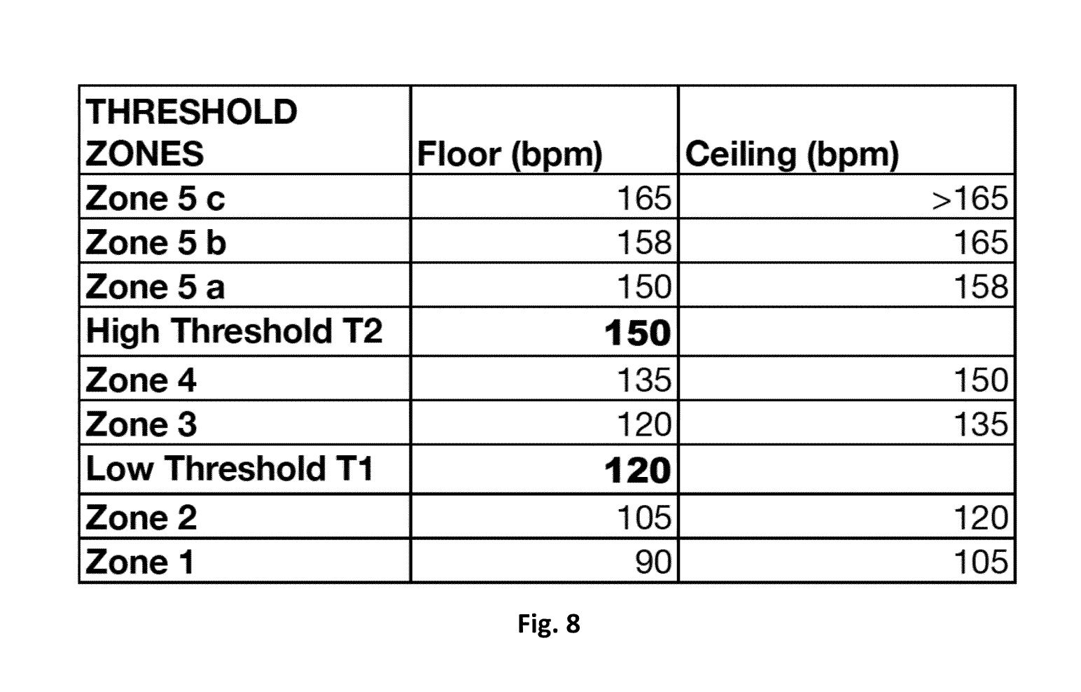

Following the general guidelines from FIG. 6, FIG. 8 depicts a detailed chart comprising seven zones. Zones 1 and 2 are below T1, while Zones 3 and 4 are between T1 and T2, and Zones 5A, 5B, and 5C are above T2.

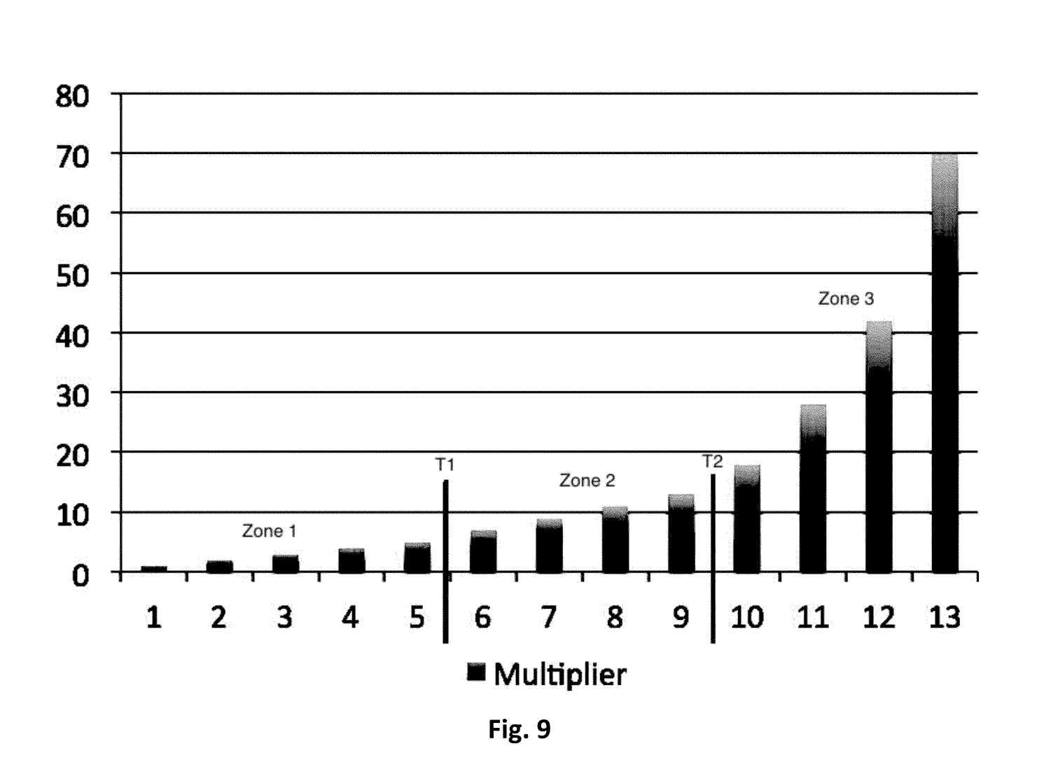

FIG. 9 shows a breakdown of zones that utilizes even more detailed zones, and is according to the preferred embodiment of the invention. Here, Zones 1-5 are shown below T1, and are shown to have a multiplier increasing linearly per zone. The multiplier corresponds roughly to oxygen consumption or power in watts and is simply a parameter of exercise stress. Between T1 and T2, the rate of change of the multiplier per unit Zone is higher, as shown in the graph by a linear (but steeper) increase from between Zones 5-9. Beyond Zone 9 the rate of change per Zone of the multiplier becomes substantially exponential as shown on the graph. Mathematically, the rate of change of NUMBER multiplier below T1 and between T2 is non-exponential, that is f(x)=nX. The value of n between T1 and T2 is some number greater than the value of n below T1. Above T2 X begins to increase exponentially, that is, where the exponent of X is some number greater than 1, or F(x)=nX^{z}, where Z is an exponent greater than the number 1.

Continuing with FIG. 9, it is also noted that the number along the Y-axis may also be used as a multiplier to determine total training load based on time. For instance, if an individual spent 10 minutes in zone 7 and 20 minutes in Zone 9, the number calculated would be 10×9+20×13, or a total 330 points. It should also be noted that FIG. 9 is but one example of the application of a multiplier and as shown throughout this application, and that fewer or more zones could be used. For instance, the number of zones leading up to the first anchor point need not necessarily be four. For instance, in an alternative embodiment of the invention, the applicant has eight zones between each anchor point for a total of 24 zones. As before, training load may be calculated by calculating the amount of time (preferably minutes, although other suitable units such as day, hours or seconds may be used) by the zone number.

As described above the system comprises a display device for the user, preferably in the form of a wrist band worn by the user. The system communicates with the display and can display to the user whether the user is below T1, between T1 and T2, or above T3. Although various means of display may be used, a preferably display blinks blue to indicate the user is below T1, blinks yellow to indicate the user is between T1 and T2, and blinks red to indicate the user is above T3. The means of communicating the zone to the user may also be audible, such as a beep that occurs at some frequency when the user is between T1 and T2, and alternatively a different frequency beep that occurs when the user is below T1 or above T2.

Different health and fitness and sports performance benefits are associated with each Zone. The benefits an individual receives while in a higher Zone are not necessarily duplicated when one is in a lower intensity Zone, and vice versa. That is, one does not receive the same benefits from Zone 1 that one would receive training in Zone 4. This is irrespective of the number of Zones the user continuous of exercise is broken down into. Said again, the benefits a user experiences from a higher Zone are qualitatively different, not quantitatively different from the benefits received in a lower zone. In the high threshold Zones, the result of a person's exercise is the building up of tolerance to high acidosis resulting from high lactate production and removal. In the lower threshold zones one is exercising to increase the capillary density and mitochondrial density in the muscle cells.

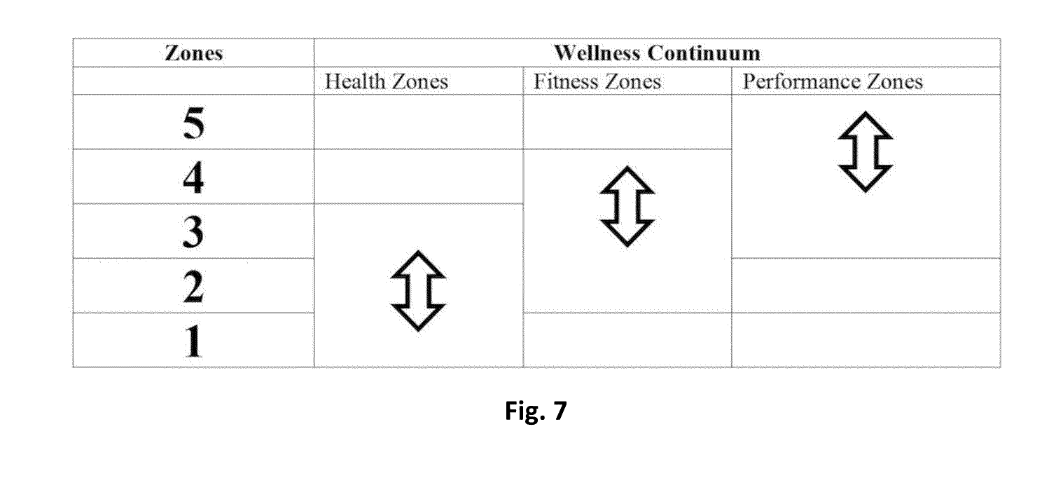

Turning now to FIG. 7, an embodiment is presented wherein five zones are utilized. Zones 1-3 are referred to collectively as the health zones, because when exercising in this zone the user primarily achieves health benefits such as better sleep at night, increased energy levels, decreased blood pressure levels, improved cholesterol levels and improved response to stress. Zones 2-4 are referred to collectively as the fitness zones, because when exercising in this zone the user primarily achieves fitness benefits such as lower fat levels, healthier metabolism, increased endurance, and increased ability to process oxygen. Zones 3-5 are collectively referred to as the performance benefits because of the performance benefits achieved through exercising within this zone. Performance benefits may include such benefits as increased top speed, increased tolerance to lactic acid buildup, increased ability to sustain high levels of oxygen consumption, and increased VO₂ max. To emphasize the fact that each individual's T1 and T2 anchor points are person-specific, they are now shown on FIG. 7. Testing might reveal that the individual's T1 for one sport lies between Zone 2 and 3, while for another sport it mitt fall squarely in Zone 2. Likewise, another individual might have a T1 anchor point somewhere different. In practice, the system may print a chart such as FIG. 7 and the user may enter in her T1 and T2 points after testing.

As there is obviously a great deal of overlap within and among the collective groupings of the zones, and the grouping should now clearly be understood to be a convenience measure for the user, the simplicity of FIG. 7 is to quickly remind the user that in general, a workout that varies between Zones 2, 3, and 4 will primarily be achieving fitness goals while a workout that varies between Zones 1, 2, and 3 will primarily be achieving health benefits.

As described above with respect to exemplary FIG. 9, one step of the Applicant's system involves the concept of training load. Training load is an important concept because it allows an individual to track his or her training performance over time. As discussed, training load is calculated by multiplying the amount of time an individual spends in a particular training zone by the multiplier associated with that training zone. By assigning multipliers that increase substantially exponentially with each zone from T2 and above, the physiological effects of higher intensity activities is properly accounted for.

In short, training load may be calculated as intensity (as determined by the Applicant's multipliers)×frequency×time. Additional factors could readily be added such as an additional activity-specific multiplier because training load calculations are activity specific depending on the movements required and the muscle groups engaged to standardize the training load value between different activities. For instance, the training load value obtained could be multiplied by 1.2 if the activity was swimming because of the external resistance of water and other factors and by 0.8 if the activity was cycling.

For purposes of demonstration, multipliers of 1, 2, 3, 4, 5, 6, 8,and 11 were selected; conforming to the Applicant's method is shown in Table 2, below. Here, 240 minutes in zone three under yields a total training load for that session of 720 points, and the 120 minutes spent in zone 4 equates to 480 points. The total training load for the week is 1680 points. Multiplying 1680 points for this week by 10 weeks at a frequency of 1 time per week would yield a training load for the ten weeks of 16,800 points.

TABLE 2
Calculating Training load in the threshold training system

| | | | | | | | | | | Total |
|---|---|---|---|---|---|---|---|---|---|---|
| Preparation 1 | Endurance Base | 20% | 30% | 40% | 20% | — | — | — | — | 100% |
| TIME IN | 10 hours × 60 min = | 120 min. | 180 min | 240 min | 120 min | — | — | — | — | 600 |
| ZONE | 600 weekly minutes x | | | | | | | | | |
| Multiplier | Heart Zones Training | 1 | 2 | 3 | 4 | 5 | 6 | 8 | 11 | |
| or ZONE | Point (Zone weight) | | | | | | | | | |
| Multipler | multiplier | | | | | | | | | |
| LOAD | Internal Training load | 120 | 360 | 720 | 480 | | | | | 1,680 |

The following table was generated using a set of multiplier values following the same substantially exponential increase as defined above and shown in Table 2, but with different values associated with the multipliers.

TABLE 3
Calculating Training load in the threshold training system

| | | | | | | | | | |
|---|---|---|---|---|---|---|---|---|---|
| Preparation 1 | Endurance Base | 20% | 30% | 40% | 20% | — | — | — | — |
| TIME IN | 10 hours × 60 min = 600 | 120 min. | 180 min | 240 min | 120 min | — | — | — | — |
| ZONE | weekly minutes x | | | | | | | | |
| POINTS | Heart Zones Training Point | 3 | 6 | 9 | 12 | 15 | 19 | 25 | 35 |
| | (Zone weight) multiplier | | | | | | | | |
| LOAD | Internal Training load | 120 | 360 | 720 | 480 | | | | |

If necessary, from this training load determination step, periodization as known in the art may be performed. Periodization is essentially a training load value sequenced and distributed over many weeks, months, or years, and refers to the distribution and sequencing of training load, as shown above. To distribute workload over weeks or months of training and to sequence it with appropriate weighting and unweighting the training needs to be quantifiable. Only recently has this quantification become possible, through the use of new tools like heart rate monitors, distance monitors, altitude monitors, power meters, and speed monitors. With these types of tools it has recently become possible for both amateur exercise enthusiasts and professional athletes to easily assess the amount of stress and load that one is experiencing.

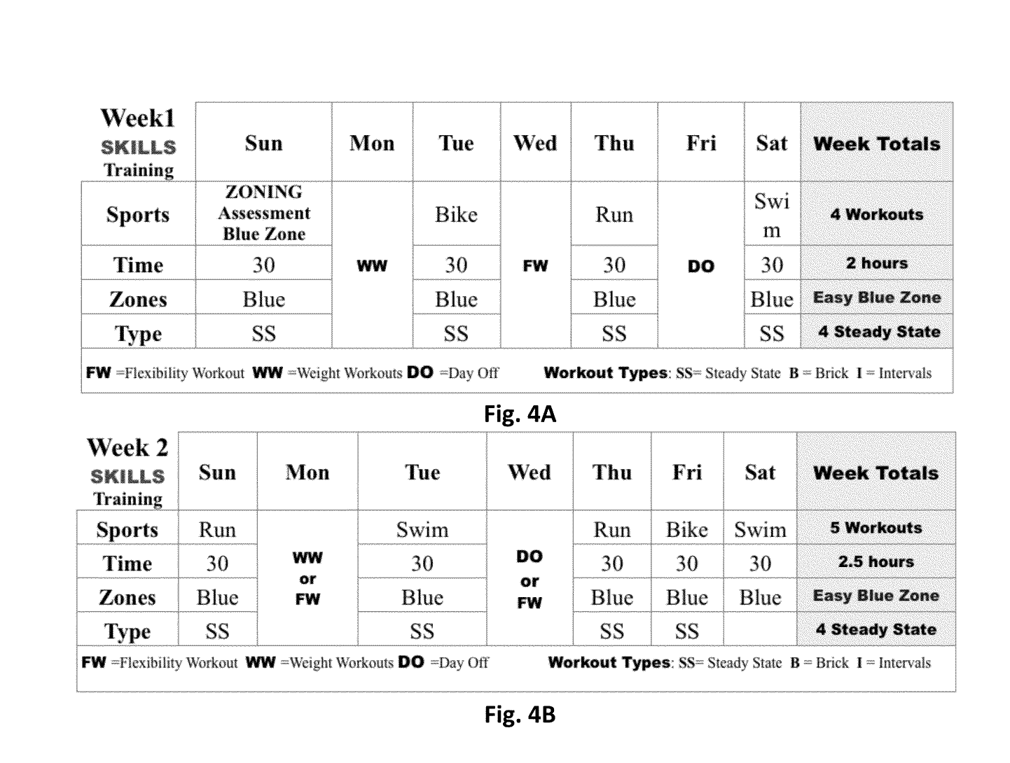

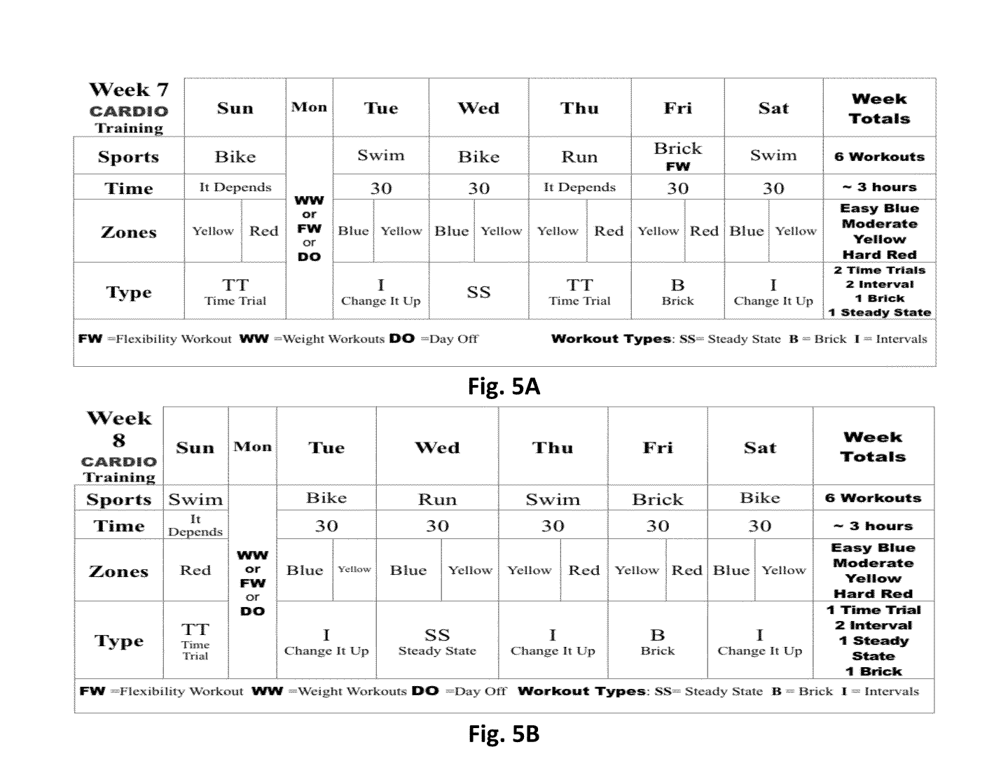

Another embodiment of the invention is shown in FIGS. 4A, 4B, 5A, and 5B. The system described herein, referred to as zoning, is a training methodology that may be used as the foundation for creating original and sport-specific training programs for both individuals and groups. FIG. 4A is an example of a printed card showing various activities and lengths of time and intensity with which they should be performed. This 7-day program shows the sports activities of swimming, cycling and running because this program is designed for a first-time triathlete. It should be appreciated that other suitable programs could be designed based on the needs of other athletes. In this exemplary case, for each day the number of minutes is included for that day as well as the zone that is the focus of the workout. For example, on Week 2 skills on Tuesday, (FIG. 4B), the participate would be focusing on a swim workout for 30 minutes in the low and easy blue zone.

The type of workout is also included because there are different ways to perform the workout. For instance, although for week 1 and 2 every workout is an SS, or steady state workout, for week 7 and 8 (FIG. 5A and 5B, respectively) TT (time trial), B (Brick) and I (Interval, Change it Up) are utilized, where in this case SS is a workout in which one's heart rate or effort is held constant with little to no variation in intensity throughout the workout time, while “Brick” is a bike-run or swim-bike session inside any single workout session. Time Trial is a tempo or at or near race effort heart rate or speed and Interval is hard effort or heart rate followed by an easy effort or lower heart rate usually based on time ratios such as 1 minute hard and 1 minute easy.

Week Totals are included as a summary of the four headings: Sport, Time, Zones, and Type. The purpose of the week total is to serve as an overview of the total 7-day workout and what's involved in total number of workouts, total hours of activity, what zones or intensities are in the program and a summary of the workout types. In FIG. 5A titled “Cardio Training” the focus of the 7 day training period is dedicated to improving the participants overall cardio endurance. Again, in the Zoning methodology, the emphasis on the training period is on the individual's heart rate as a way to assess exercise effort or training load or work load. The workouts in FIGS. 5A and 5B are more challenging with higher intensities that include all three color zones in the ZONING methodology: low Blue zone training, moderate Yellow zone training, and high and hot Red zone workouts.

The Applicant has found through experimentation that fitness levels increase optimally when an individual spends time within each different heart zone. Sports periodization plans, such as those dating back to Selye's General adaptation system from the late 1950s, may be adapted to include the T1 and T2 anchor points disclosed here and associated multipliers disclosed by the applicant.

Periodization plans may be personalized, should be variable (to prevent training monotony and to stimulate positive effects from training), should be planned according to the amount of time an individual expects to have for training, and should be logged. Accurate logging is critical in gauging the effectiveness of a training program. In particular, details about the training and about the context of one's life in which the training occurs should be logged. Recovery or regeneration time should also be built into any periodization plan.

In use, a fitness enthusiast using the Applicant's training system should not measure or gauge his or her workout based on the number of beats per minute at which she is training Instead, the user should think of training as a certain percentage of the T1 anchor point, or, in an alternative embodiment of the invention, as a certain percentage of the HRₘₐₓ.

The steps outlined above, i.e. determining the first and second anchor point using a means such as the Talk Threshold test allows T1 and T2 to be determined for the individual and for the individual's sport such that as shown in FIG. 6 the information is personalized to the user.

Once the user of the Applicant's system has determined his or her own personalized zone fitness information; it is ideally printed in the form of a chart that may be easily accessible during the user's physical activity. Information about the current zone an exerciser is in may also be relayed to the user through a display either worn on the user's wrist or otherwise placed within the sight of the exerciser. FIGS. 7 and 8 depict sample charts created using threshold system while FIGS. 1 and 2 depict sample wrist displays. Note that except for the highest zone, the ceiling of one zone corresponds closely or exactly to the floor of the zone above it.

In an additional alternative embodiment of the present application, multiplier values and data regarding T1 and T2 are stored in a computer-readable medium for display on a device such as a personal computer, mobile telephone (including telephone/PDA devices), or application (app) for a mobile device. In alternative display information concerning this data is displayed to the user via an eyeglasses mounted display or other heads up display or via a display associated with a cardiovascular exercise machine. The display system on said devices in these alternative embodiments display information regarding the first and second anchor points and associated zones for an individual. For instance, FIGS. 1 and 2 above are suitable examples for this display. The computer-readable medium further stores instructions to determine a personalized heart rate range for each of said zones, wherein the range is a percentage of the first anchor point heart rate. The means for determining training load as described above could without difficulty be implemented by a computer program or app for a mobile device.

The foregoing description of the preferred embodiment of the present invention has been presented for the purpose of illustration and description. It is not intended to be exhaustive or to limit the invention to the precise form disclosed. Many modifications and variations are possible in light of the above teachings. For example, the appearance of the watch or display means may be different depending on the personal tastes of the wearer or the sports activities in which the wearer is involved. Moreover, in some embodiments, the system may include fewer components or additional components. It is intended that the scope of the present invention not be limited by this detailed description, but by the claims and the equivalents to the claims appended hereto.

I claim:

1. A method for measuring and reporting exercise stress of an individual, the method comprising:
a. measuring a heart rate level of an individual while the individual exercises, and calculating therefrom a first anchor point and a second anchor point;
b. providing:
i. a computer readable memory for storage of said anchor points;
ii. a processor in communication with said memory that contains instructions that determine a plurality of zones, at least two anchor points, and a plurality of heart rate ranges, wherein each said heart rate range corresponds to one of said plurality of zones, and where each of said plurality of zones has associated thereto a multiplier;
iii. a timer; and
iv. a display capable of displaying at least three colors;
c. wherein said heart rate level is within said heart rate range for an amount of time recorded by said timer;
d. multiplying said multiplier by said amount of time to calculate a subtotal;
e. repeating said multiplying step a plurality of times;
f. adding said subtotals to calculate a training load;
g. displaying to a user information regarding said training load; and
h. displaying to the user with said display at least one of said at least three colors, the at least one color displayed corresponding to said heart rate range.

2. The method of claim 1 further comprising printing on paper a chart comprising said information.

3. The method of claim 2 wherein said chart comprises major subdivisions that correspond to each of said plurality of zones.

4. The method of claim 3 wherein one of said major subdivisions is further divided into a plurality of minor subdivisions.

5. The method of claim 3 wherein each of said major subdivisions corresponds to a color.

6. The method of 1 wherein for zones above said first anchor point said multiplier increases at a faster rate per zone than for zones below said anchor point, and where for zones above said second anchor point said multiplier increases at a substantially exponential rate per zone.

7. The method of claim 1 wherein said at least three colors are blue, yellow, and red light to the user wherein a blue light corresponds to a heart rate level below said first anchor point, a yellow light corresponds to a heart rate level above said first anchor point but below said second anchor point, and a red light corresponds to a heart rate level above said second anchor point.

8. A method of displaying to a user via a display device worn on the user's body data regarding a personalized set of intensity zones for the user, the method comprising the steps of:
a. determining a first anchor point corresponding to a first heart rate of an individual as the individual partakes in an activity, wherein said first anchor point represents a first change in slope of an individual's heart rate wherein below said first anchor point said slope increases substantially linearly at rate X and above said first anchor point said slope increases substantially linearly at at >X;
b. determining a second anchor point corresponding to a second heart rate of said individual as the individual partakes in said activity, wherein said second anchor point represents a second change in slope of an individual's heart rate wherein said slope changes from increasing substantially linearly to substantially exponentially;
c. providing:
i. a computer readable memory for storage of said anchor points; and
v. providing a processor in communication with said memory and that contains instructions that generate based on said anchor points a personalized heart rate range for each of a plurality of heart rate intensity zones, and wherein each said intensity zone corresponds to a multiplier; and
d. displaying to a user via a user-worn display data regarding said intensity zones.

9. The method according to claim 8 wherein each said intensity zone comprises major subdivisions, and one of said major subdivisions is further divided into a plurality of minor subdivisions.

10. The method according to claim 9 wherein each of said major subdivisions corresponds to a color.

11. The method of 8 wherein said heart rate of said individual is within said heart rate range for an amount of time, further comprising the step of multiplying said amount of time by said multiplier to determine a training load.

12. The method of claim 8 wherein said computer readable memory and said processor are components of a mobile telephone.

13. A computer-readable medium containing instructions for controlling a computing device to generate a personalized heart rate range for an individual, said instructions comprising:
a. instructions to input a plurality of anchor points based on a heart rate of an individual; and
b. instructions to determine said personalized heart rate range as a percentage of said anchor points for each of a plurality of heart rate intensity zones, wherein each said intensity zone corresponds to a multiplier, wherein said heart rate range comprises an upper heart rate and a lower heart rate, and wherein one of said plurality of heart rate intensity zones is a threshold zone.

14. The computer-readable medium of claim 13 further comprising instructions wherein said individual's heart rate is within said heart rate range for an amount of time, said computer-readable medium further comprising:
a. instructions to record said amount of time; and
b. instructions to multiply said amount of time by said multiplier to determine a training load.

15. The computer-readable medium of claim 13 wherein said computing device comprises a mobile telephone.
